(19) United States
(12) Patent Application Publication
(10) Pub. No.: US 2024/0102075 A1
(43) Pub. Date: Mar. 28, 2024

(54) METHOD FOR QUANTIFYING 25-HYDROXYVITAMIN D, HYDROXYLASE, QUANTIFICATION COMPOSITION, QUANTIFICATION KIT, ELECTRODE, SENSOR CHIP, AND SENSOR

(71) Applicant: KIKKOMAN CORPORATION, Noda-shi (JP)

(72) Inventors: Kanako HAYASHI, Noda-shi (JP); Atsushi ICHIYANAGI, Noda-shi (JP)

(21) Appl. No.: 18/538,143

(22) Filed: Dec. 13, 2023

Related U.S. Application Data
(63) Continuation of application No. PCT/JP22/23533, filed on Jun. 10, 2022.

(30) Foreign Application Priority Data
Jun. 15, 2021 (JP) 2021-099705

(51) Int. Cl. C12Q 1/26; G01N 27/327

(52) U.S. Cl. CPC C12Q 1/26; G01N 27/327

(57) ABSTRACT

A novel method for quantifying a 25-hydroxyvitamin D concentration, a hydroxylase, a quantification composition, a quantification kit, an electrode, a sensor chip, and a sensor are provided. A method for quantifying 25-hydroxyvitamin D by adding a mediator and hydroxylase to a sample can be provided. Quantification of an oxidized mediator produced by an action of the hydroxylase, or oxygen or a reduced mediator consumed by an action of the hydroxylase may be included.

### CROSS-REFERENCE TO RELATED APPLICATIONS

This application is a Continuation of International Patent Application No. PCT/JP2022/023533, filed on Jun. 10, 2022, which claims the benefit of priority to Japanese Patent Application No. 2021-99705, filed on Jun. 15, 2021, the entire contents of which are incorporated herein by reference.

### REFERENCE TO AN ELECTRONIC SEQUENCE LISTING

The contents of the electronic sequence listing (named “TAKA-0034US1_KM21001WUS0_Sequence Listing filed”; Size: 14 KB; and Date of Creation: Dec. 13, 2023) is herein incorporated by reference in its entirety.

### FIELD

The present invention relates to a method for quantifying 25-hydroxyvitamin D, a hydroxylase, a quantification composition, a quantification kit, an electrode, a sensor chip, and a sensor.

### BACKGROUND

Vitamin D is a physiologically active substance that acts on various biological processes in the body. Vitamin D is divided into vitamin D2 (Ergocalciferol) and vitamin D3 (Cholecalciferol). The sources of vitamin D are the biosynthesis of vitamin D3 at the skin and the ingestion of vitamin D2 and vitamin D3 from foods and supplements. Since vitamin D2 and vitamin D3 undergo the same metabolism and have the same effect, they are referred to as vitamin D when not distinguishing between vitamin D2 and vitamin D3.

Vitamin D taken into the body is hydroxylated in the liver and converted to 25-hydroxyvitamin D, which is stored in hepatocytes. 25-hydroxyvitamin D binds to a vitamin D-binding protein and is released into the blood. Since the blood half-life of 25-hydroxyvitamin D is as long as about 2 to 3 weeks, it is believed that blood 25-hydroxyvitamin D concentration is an index reflecting the vitamin D concentration in the body.

25-hydroxyvitamin D in the blood is taken into cells by endocytosis via the megalin receptor. The 25-hydroxyvitamin D transferred to the renal tubules of the kidney is hydroxylated and converted to the active form of 1,25-dihydroxyvitamin D. The 1,25-dihydroxyvitamin D, which is released back into the blood, binds to vitamin D receptors within a target cell and acts as a transcription factor that controls the expression of various types of genes related to calcium transport and utilization. The half-life of 1,25-dihydroxyvitamin D in the blood is as short as about 15 hours, and the concentration of 1,25-dihydroxyvitamin D in the blood is tightly controlled by a parathyroid hormone, calcium, and phosphate. Therefore, it is believed that the blood level of 1,25-dihydroxyvitamin D does not change unless there is an extreme deficiency of or excess vitamin D.

Vitamin D plays an essential role in controlling calcium and phosphate concentrations in the body. Vitamin D mainly affects intestinal cells and osteocytes, assisting in the control of calcium uptake in the former and in the formation and maintenance of the skeleton in the latter. In addition, vitamin D is known to be involved in cell proliferation, cell differentiation and the immune system. A deficiency of or excess vitamin D has various consequences for the body. In particular, vitamin D deficiency may lead to serious diseases such as rickets, osteomalacia, osteoporosis, chronic renal failure, hyperparathyroidism, and psoriasis.

The criteria for the determination of vitamin D insufficiency or deficiency are that a serum 25-hydroxyvitamin D concentration of 30 ng/ml or more is considered vitamin D sufficient, 20 ng/ml or more or less than 30 ng/ml is considered vitamin D insufficiency, and less than ng/ml is considered vitamin D deficiency (according to Assessment Criteria for Vitamin D Deficiency/Insufficiency by The Japanese Society for Bone and Mineral Research and The Japan Endocrine Society). The number of patients with vitamin D deficiency is currently estimated to be 1 billion worldwide. In particular, early detection of vitamin D deficiency is useful in modern society, where direct sunlight is likely to be avoided. In Japan, electrochemiluminescence immunoassay (ECLIA method), chemiluminescence enzyme immunoassay (CLEIA method), and chemiluminescence immunoassay (CLIA method) of 25-hydroxyvitamin D in a serum, and the like are insured. These are all immunological measurement methods using an anti-25-hydroxyvitamin D antibody.

For example, Japanese laid-open patent publication No. 2017-40659 discloses an antibody or an antigen-binding fragment thereof that recognizes 25-hydroxyvitamin D and a method for measuring 25-hydroxyvitamin D using the same. The measurement of 25-hydroxyvitamin D using the anti-25-hydroxyvitamin D antibody has high sensitivity with the detection limit of 25-hydroxyvitamin D of less than 3.0 ng/ml, and it is considered to be useful for early diagnosis of vitamin D deficiency. However, since the method is an examination method using the immunological measurement method, there is also a problem that the time required for the measurement is long and the examination cost is high.

Therefore, a method for measuring 25-hydroxyvitamin D itself, rather than the immunological measurement method, is desired. For example, U.S. Pat. No. 5,981,779 discloses a method for measuring 25-hydroxyvitamin D by competitive binding to a vitamin D binding protein using biotin- or fluorocein-labeled 25-hydroxyvitamin D. Japanese laid-open patent publication No. 2009-540275 discloses a method for measuring 25-hydroxyvitamin D using high performance liquid chromatography (HPLC). Japanese laid-open patent publication No. 2018-81023 discloses a method for measuring 25-hydroxyvitamin D using a liquid chromatography-mass spectrometer (LC/MS/MS).

### SUMMARY

However, the above-described method for measuring 25-hydroxyvitamin D has various drawbacks such as long measurement times, errors in measurement values, large costs, sample volumes, and reagents which are difficult to handle, and is not optimal for clinical testing. Therefore, there is a need for a method for measuring 25-hydroxyvitamin D that is less laborious, time consuming, and costly.

On the other hand, International patent publication No. WO2007/138894 discloses a vitamin D3 hydroxylase of Pseudonocardia autotrophica.

One of the objects of the present invention is to provide a new quantitation method, a hydroxylase, a quantification composition, a quantification kit, an electrode, a sensor chip, and a sensor.

According to an embodiment of the present invention, a method for quantifying 25-hydroxyvitamin D by adding a mediator and hydroxylase to a sample is provided.

An oxidized mediator produced by an action of the hydroxylase, or oxygen or a reduced mediator consumed by an action of the hydroxylase may be quantified.

According to an embodiment of the present invention, hydroxylase used in the method for quantifying 25-hydroxyvitamin D is provided.

According to an embodiment of the present invention, a composition for quantifying 25-hydroxyvitamin D containing hydroxylase is provided.

According to an embodiment of the present invention, a kit for quantifying 25-hydroxyvitamin D including hydroxylase and a mediator oxidized by adding hydroxylase is provided.

According to an embodiment of the present invention, an electrode containing hydroxylase is provided.

According to an embodiment of the present invention, a sensor chip having an electrode as a working electrode is provided.

According to an embodiment of the present invention, a sensor having a sensor chip is provided.

### BRIEF DESCRIPTION OF DRAWINGS

FIG. 1A is a schematic diagram of a sensor chip 10 according to an embodiment of the present invention.

FIG. 1B is a schematic diagram showing a member constituting a sensor chip 10 according to an embodiment of the present invention.

FIG. 1C is a schematic diagram showing a member constituting a sensor chip 10 according to an embodiment of the present invention.

FIG. 1D is a schematic diagram showing a member constituting a sensor chip 10 according to an embodiment of the present invention.

FIG. 2A is a schematic diagram of a sensor 100 according to an embodiment of the present invention.

FIG. 2B is a block diagram of a sensor 100 according to an embodiment of the present invention.

FIG. 3 is a graph showing a relationship between a 25-hydroxyvitamin D concentration and an amount of change in absorbance (ΔA666) according to Example 1 of the present invention.

FIG. 4 is a diagram showing a reaction time of 25-hydroxyvitamin D and an amount of change in absorbance (A340) according to Example 2 of the present invention.

### DESCRIPTION OF EMBODIMENTS

Hereinafter, a novel quantitation method for measuring 25-hydroxyvitamin D, a hydroxylase, a quantification composition, a quantification kit, an electrode, a sensor chip, and a sensor according to the present invention will be described. However, the novel quantitation method for measuring 25-hydroxyvitamin D, the hydroxylase, the quantification composition, the quantification kit, the electrode, the sensor chip, and the sensor of the present invention are not to be construed as being limited to the description contents of the embodiments and examples described below.

In an embodiment, a hydroxylase used in the present invention is a hydroxylase acting on the substrate 25-hydroxyvitamin D. An enzyme capable of directly hydroxylating 25-hydroxyvitamin D is disclosed in International patent publication No. WO2007/138894. The present inventors have found that Vitamin D hydroxylase (VDH) derived from a Pseudonocardia autotrophica NBRC12743 strain is useful for quantifying 25-hydroxyvitamin D.

In the present specification, a hydroxylase derived from a Pseudonocardia autotrophica NBRC12743 strain is shown and described as an exemplary hydroxylase, but the present invention is not limited to this, and may include those having hydroxylation reactivity to 25-hydroxyvitamin D of a certain level or more. For example, among the hydroxylases belonging to EC No. 1.14, an enzyme that recognizes 25-hydroxyvitamin D as a substrate and has 25-hydroxyvitamin D hydroxylase activity can be used as the hydroxylase.

In an embodiment, the hydroxylase may be a hydroxylase produced by a naturally occurring microorganism or a hydroxylase produced by a transformed microorganism. From the viewpoint of efficient mass expression of the enzyme, the enzyme can be efficiently mass-expressed by using the transformed microorganism.

In an embodiment, the hydroxylase may be multimeric or monomeric. For example, in the case of catalyzing a hydroxylation reaction in which an oxygen atom is added to a substrate and a hydroxylation group is formed by only a certain subunit (monomer) among several subunits constituting a hydroxylase which is a multimer, the hydroxylase used in the present invention may be a multimer or a subunit (monomer). In addition, as long as it has 25-hydroxyvitamin D hydroxylase activity, it may be composed of a partial structure of an enzyme.

As described above, the present inventors have found that vitamin D hydroxylase derived from a Pseudonocardia autotrophica NBRC12743 strain is useful for quantifying 25-hydroxyvitamin D, which is a vitamin D derivative. In an embodiment, the hydroxylase according to the present invention includes a hydroxylase derived from a Pseudonocardia autotrophica NBRC12743 strain, but also a hydroxylase derived from microorganisms classified in the class Actinomycetes, and also a hydroxylase derived from microorganisms classified as Pseudonocardia autotrophica. In an embodiment, the hydroxylase of the present invention may be a hydroxylase derived from microorganisms classified as Pseudonocardia ammonioxydans, Pseudonocardia parietis, Pseudonocardia alni, Pseudonocardia antarctica, Pseudonocardia sediminis, Pseudonocardia pini, Pseudonocardia acidicola, and Pseudonocardia broussonetiae. In addition, a hydroxylase which has a high sequence identity (e.g., 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, and 99% or more) relative to the amino acid sequence of the hydroxylase in SEQ ID NO: 1 and a hydroxylase having an amino acid sequence in which one or more amino acids are altered or varied, deleted, substituted, added and/or inserted in the amino acid sequence of SEQ ID NO: 1. Further, the hydroxylase can also be screened by culturing microorganisms of the Pseudonocardia autotrophica NBRC12743 strain under a predetermined condition (for example, see Japanese Society for Bacteriology Journal, 18 (1), 1963), mixing a hydroxylase reaction reagent containing 25-hydroxyvitamin D (details are described below) with an extract obtained by disrupting the bacterial cells, and confirming the presence or absence of reactivity with the reagent.

In an embodiment, the present invention provides DNA encoding of a hydroxylase. In an embodiment, the present invention provides DNA encoding of the amino acid sequence shown in SEQ ID NO: 1, or DNA having the base sequence shown in SEQ ID NO: 2. In an embodiment, the present invention provides DNA encoding of a protein having a base sequence of 40% or more, 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more relative to the base sequence shown in SEQ ID NO: 2, and having 25-hydroxyvitamin D hydroxylase activity. In an embodiment, the hydroxylase of the present invention may be a hydroxylase derived from a Pseudonocardia autotrophica NBRC12743 strain or a hydroxylase produced by Escherichia coli transformed with a plasmid containing a hydroxylase gene derived from a Pseudonocardia autotrophica NBRC12743 strain, but by using the E. coli transformed with a plasmid containing a hydroxylase gene derived from a Pseudonocardia autotrophica NBRC12743 strain, hydroxylase can be efficiently expressed in large quantities.

In an embodiment, any conditions may be used as the hydroxylase reaction condition as long as the condition acts on 25-hydroxyvitamin D and efficiently catalyzes the hydroxylation reaction. Generally, enzymes have an optimum temperature and pH at which the enzymes exhibit the highest activity. Therefore, the reaction condition is preferably near the optimum temperature and the optimum pH. In an embodiment, the reaction condition of the hydroxylase includes a method for quantifying 25-hydroxyvitamin D under a condition other than the optimum condition of the enzyme alone by comprehensively examining conditions based on a composition other than the enzyme, for example, conditions suitable for components such as a chromogenic reagent, a mediator, an enzyme stabilizer, and a stabilizer of a measurement sample, and compatibility with a measurement device.

### [Vector]

For example, any vector known to a person skilled in the art such as a bacteriophage or a cosmid can be used as the vector that can be used in the present invention. Specifically, for example, pUC18 (manufactured by Takara Bio Inc.), pBluescriptII SK+(manufactured by Agilent Technologies Inc.), pET-16b, pET-22b(+) (manufactured by Merck), and the like are preferable.

### [Construction of Plasmid for Expression]

The plasmid for hydroxylase expression according to the present invention is obtained by a commonly used method. For example, DNA is extracted from a microorganism producing the hydroxylase according to the present invention to create a DNA library. From the created DNA library, a DNA fragment encoding the hydroxylase according to the present invention is identified and isolated. The DNA fragment is amplified by a polymerase chain reaction (PCR) with complementary primers using the isolated DNA fragment as a template to clone a gene encoding the hydroxylase according to the present invention. The amplified DNA fragment is ligated into a vector to obtain a plasmid having the DNA fragment encoding the hydroxylase according to the present invention.

Alternatively, the DNA fragment encoding the hydroxylase according to the present invention is chemically synthesized, the DNA fragment is ligated into a vector, and a plasmid having DNA encoding the hydroxylase according to the present invention is obtained.

In the case where a DNA base sequence of the hydroxylase gene obtained by the above-described method is determined or confirmed, for example, Applied Biosystems 3730xl DNA analyzer (manufactured by Thermo Fisher Scientific) can be used.

### [Transformation and Transduction]

The hydroxylase gene obtained as described above can be incorporated into a vector such as a bacteriophage, a cosmid, or a plasmid used for transformation of a prokaryotic cell or a eukaryotic cell by a conventional method, and hosts corresponding to each vector can be transformed or transduced by a conventional method. For example, the obtained recombinant DNA can be used to transform or transduce any host, for example, a microorganism belonging to the genus Escherichia, specifically, an E. coli K-12 strain, preferably an E. coli JM109 strain, an E. coli DH5a strain (both manufactured by Takara Bio Inc.), an E. coli B strain, preferably an E. coli BL21 strain (manufactured by Nippon Gene Co., Ltd.), or the like, to obtain each strain.

In addition, for example, an example of a eukaryotic host cell is yeast. Microorganisms classified as yeast include, for example, yeasts belonging to the genus Zygosaccharomyces, the genus Saccharomyces, the genus Pichia, and the genus Candida. The inserted gene may include a marker gene to allow the selection of transformed cells. The marker gene includes, for example, genes which complement the auxotrophy of the host, such as URA3, TRP1. In addition, it is desirable that the inserted gene contains a promoter or other control sequence (for example, secretory signal sequence, enhancer sequence, terminator sequence, polyadenylation sequence, and the like) capable of expressing the gene of the present invention in the host cell. Specific examples of the promoter include a GAL1 promoter and an ADH1 promoter, and the like. A known method, for example, a method using lithium acetate (Methods Mol. Cell. Biol., 5, 255-269 (1995)), electroporation (J Microbiol Methods 55 (2003) 481-484), or the like can be suitably used as a method of transformation into yeast, but the transformation may be performed using any of a variety of methods including, but not limited to, a spheroplast method, a glass bead method, and the like.

For example, other examples of eukaryotic host cells include filamentous fungi such as the genus Aspergillus and the genus Trichoderma. A method for producing a transformant of a filamentous fungus is not particularly limited, and examples thereof include a method of inserting the gene encoding a hydroxylase into a host filamentous fungus in an aspect in which the gene encoding a hydroxylase is expressed according to a conventional method. Specifically, a transformant overexpressing the gene encoding a hydroxylase is obtained by making a DNA construct in which the gene encoding a hydroxylase is inserted between an expression-inducing promoter and a terminator, then transforming the host filamentous fungus with a DNA construct containing the gene encoding a hydroxylase. In this specification, the DNA fragment consisting of an expression inducible promoter—a gene encoding a hydroxylase—terminator and a recombinant vector containing the DNA fragment produced for transforming a host filamentous fungus are collectively referred to as a DNA construct.

The method of inserting the gene encoding hydroxylase into the host filamentous fungus in such a manner that the gene is expressed is not particularly limited, and for example, the method includes a method of inserting the gene directly into the chromosome of the host organism by using homologous recombination, or a method of introducing the gene into the host filamentous fungus by linking on a plasmid vector, and the like.

In a method utilizing homologous recombination, the DNA construct can be ligated between sequences homologous to an upstream region and a downstream region of the recombination site on the chromosome and inserted into the genome of the host filamentous fungus. Transformants by self-cloning can be obtained by overexpressing within the host filamentous fungi under high expression promoter control of the host filamentous fungus itself. The high expression promoter is not particularly limited, and examples thereof include a promoter region of a TEF1 gene (tef1), which is a translational elongation factor, a promoter region of an α-amylase gene (amy), and an alkaline protease gene (alp) promoter region.

In a method utilizing the vector, the DNA construct can be incorporated into the plasmid vector used in the transformation of filamentous fungi by a conventional method, and the corresponding host filamentous fungus can be transformed by a conventional method.

Such a suitable vector-host system is not particularly limited as long as it is a system capable of producing a hydroxylase in a host filamentous fungus, and examples thereof include a system of pUC19 and filamentous fungi, a system of pSTA14 (Mol. Gen. Genet. 218, 99-104, 1989), and filamentous fungi.

Although the DNA construct is preferably used by being introduced into the chromosomes of the host filamentous fungus, as another method, it can also be used without being introduced into the chromosome by incorporating the DNA construct into an autonomously replicated vector (Ozeki et al. Biosci. Biotechnol. Biochem. 59, 1133 (1995)).

The DNA construct may include a marker gene to allow the selection of transformed cells. The marker gene is not particularly limited, and examples of the marker gene include a gene which complements the auxotrophy of the host, such as pyrG, niaD, adeA, and a drug resistance gene against the drug, such as pyrithiamine, hygromycin B, or oligomycin. It is also preferred that the DNA construct contains a promoter, terminator, or other control sequences (for example, enhancer, a polyadenylation sequence, and the like) which allows for overexpression of the gene encoding hydroxylase in the host cell. Promoters include, but are not limited to, an appropriate expression-inducing promoter and a constitutive promoter, such as a tef1 promoter, an alp-promoter, an amy-promoter, and the like. The terminator is also not particularly limited, and examples thereof include an alp terminator, an amy terminator, and a tef1 terminator.

In the DNA construct, the expression control sequence of the gene encoding hydroxylase is not necessarily required when the DNA fragment including the gene encoding the hydroxylase to be inserted includes a sequence having an expression control function. In addition, when transformation is performed by a co-transformation method, the DNA construct may not have to have a marker gene.

An embodiment of the DNA construct is a DNA construct in which, for example, the tef1 promoter, the gene encoding hydroxylase, the alp terminator, and the pyrG marker gene are ligated to the In-Fusion Cloning Site at the multicloning site of the pUC19.

As a transformation method to the filamentous fungus, a method known to a person skilled in the art can be appropriately selected, and for example, a protoplast PEG method (for example, see Mol. Gen. Genet. 218, 99-104, 1989, Japanese laid-open patent publication No. 2007-222055 and the like) using polyethylene glycol and calcium chloride after preparing a protoplast of a host filamentous fungus can be used. An appropriate medium is used for regenerating the transformed filamentous fungus depending on the host filamentous fungus to be used and the transformation marker gene. For example, when Aspergillus sojae is used as the host filamentous fungus and a pyrG gene is used as the transformation marker gene, regeneration of the transformed filamentous fungus can be performed, for example, in a Czapek-Dox minimal medium (manufactured by Difco laboratories) containing 0.5% agar and 1.2 M sorbitol.

### [Identity or Similarity of Amino Acid Sequence]

The identity or similarity of an amino acid sequence can be calculated by programs such as maxim matching and search homology of GENETYX Ver.11 or Ver.14 (manufactured by GENETYX CORPORATION) or programs such as maxim matching or multiple alignment of DNASIS Pro (manufactured by Hitachi Solutions, Ltd.) or the like. When the amino acid sequences of two or more hydroxylases are aligned in order to calculate the sequence identity of an amino acid, a position of the amino acids which is identical in the two or more hydroxylases can be examined. An identical region in the amino acid sequence can be determined based on such information.

In addition, a position of the amino acid which is similar in two or more hydroxylases can also be determined. For example, CLUSTALW can be used to align a plurality of amino acid sequences, where Blosum62 is used as an algorithm and the amino acid which is determined to be similar when the plurality of amino acid sequences is aligned may be referred to as a similar amino acid. In the variants of the present invention, amino acid substitutions may be due to substitutions between such similar amino acids. Such alignment allows for examination of regions where the amino acid sequence is the same and positions occupied by the similar amino acids for the plurality of amino acid sequences. Regions of homology (conserved regions) in the amino acid sequence can be determined based on such information.

### [Method for Preparing Enzyme]

Hereinafter, a method for preparing a hydroxylase according to the present invention will be described.

The strain such as E. coli is transformed with a plasmid having the DNA encoding the hydroxylase according to the present invention, and the strain such as E. coli having the DNA encoding the hydroxylase according to the present invention is obtained.

### [Recombinant Expression of Enzyme]

The strain such as E. coli having the DNA encoding the hydroxylase according to the present invention is cultured in the medium. When culturing the microbial host cell, it may be carried out by aeration-agitated deep culture, shaking culture, stationary culture, or the like, at a culture temperature of 10 to 42° C., preferably at a culture temperature of about 25° C., for several hours to several days, and more preferably at a culture temperature of about 25° C., for 1 to 7 days. Any synthetic medium and natural medium can be used as long as it contains an appropriate proportion of the normal medium for culturing filamentous fungi, that is, a carbon source, a nitrogen source, an inorganic substance, and other nutrients. In addition, as the medium for culturing the microorganism host cell, for example, a medium is used in which one or more kinds of inorganic salts such as sodium chloride, monopotassium phosphate, dipotassium phosphate, magnesium sulfate, magnesium chloride, ferric chloride, ferric sulfate, or manganese sulfate are added to one or more kinds of nitrogen sources such as yeast extract, tryptone, peptone, meat extract, corn steep liquor, or leaching solution of soybean or wheat bran, and if necessary, a saccharine material, vitamins, and the like are appropriately added.

Culture conditions of filamentous fungi generally known by those skilled in the art may be adopted, for example, the initial pH of the medium may be adjusted to 5 to 10, the culture temperature may be set to 20° C. to 40° C., and the culture time may be set to several hours to several days, preferably 1 to 7 days, more preferably 2 to 5 days, and the like. The culturing means is not particularly limited, and aeration-agitated deep culture, shaking culture, stationary culture, or the like can be employed, but it is preferred to culture under conditions such that dissolved oxygen becomes sufficient. For example, an exemplary medium and culture conditions for culturing a microorganism of the genus Aspergillus include a shaking culture at 30° C. and 160 rpm for 3 to 5 days using a DPY medium.

After completion of the culture, the hydroxylase of the present invention is collected from the culture. For this procedure, a conventional known enzyme collection means may be used. For example, the culture medium supernatant fraction may be collected, or the bacterial cells may be subjected to ultrasonic grinding treatment, grinding treatment, or the like by a conventional method, or the present enzyme may be extracted using a lytic enzyme such as lysozyme or yatalase, or the present enzyme may be subjected to lysis by shaking or standing in the presence of toluene or the like, and the enzyme may be discharged out of the bacterial cells. Then, the solution is filtered, centrifuged or the like to remove a solid portion, and if necessary, the nucleic acid is removed by streptomycin sulfate, protamine sulfate, or manganese sulfate or the like, and then ammonium sulfate, alcohol, acetone, or the like is added thereto to fractionate, and the precipitate is collected to obtain a crude enzyme of the hydroxylase of the present invention.

### [Purification of Enzyme]

The method for purifying the enzyme may be any method as long as it is capable of purifying an enzyme from a crude enzyme solution. For example, purified hydroxylase enzyme preparations of the present invention can be obtained by appropriately selecting a gel filtration method using Sephadex, Sepharose, Superose, Sephacryl, Superdex, Ultrogel, or a biogel, and the like, an adsorption elution method using ion exchangers such as quaternary ammonium (Q), diethylaminoethyl (DEAE), diethylaminopropyl (ANX) as anion exchangers, sulfopropyl (SP), methylsulfonate (S), and carboxymethyl (CM) as cation exchangers, an adsorption elution method using hydroxyapatite, an adsorption elution method using a hydrophobic carrier such as a butyl group, a phenyl group, and an octyl group, an adsorption elution method using ligands such as iminodiacetic acid, a tresyl group, a nickel ion, a glutathione, an amylothresin, a dextrin, and streptavidin, an electrophoresis method using a polyacrylamide gel, a sedimentation method such as a sucrose density gradient centrifugation method, a fractionation method using a molecular sieve membrane or a hollow fiber membrane, or the like or performing these methods in combination.

### [Enzyme Activity Measurement]

A method for measuring the activity of the enzyme may be any method as long as it directly or indirectly measures a product or consumption in the hydroxylation reaction catalyzed by the enzyme. Generally, an enzyme has an optimum temperature and optimum pH which exhibit the highest activity. Therefore, the reaction conditions are preferably near the optimum temperature and the optimum pH.

In an embodiment, as a hydroxylase reaction process, various chemicals may be participated when the hydroxylase of the present invention acts on 25-hydroxyvitamin D. For example, electron transfer may be participated when the hydroxylase acts on 25-hydroxyvitamin D. For example, a reduced mediator may be participated when the hydroxylase acts on 25-hydroxyvitamin D.

In an embodiment, the reduced mediator supplies electrons to the hydroxylase and the hydroxylase supplied with electrons to the heme iron hydroxylates 25-hydroxyvitamin D. The mediator is oxidized by supplying electrons. Enzyme activity can be measured by quantifying the oxidized form of the mediator. For example, the mediator may be a light-absorbing substance, in which case the enzyme activity can be measured by performing an absorbance measurement. For example, the enzyme activity can be measured by reacting a reagent containing a light-absorbing substance reacting with a mediator (hereinafter, referred to as a “light-absorbing reagent”) and performing the absorbance measurement. For example, the enzyme activity can be measured by measuring a current value generated by the mediator receiving electrons from the electrode. In addition, oxygen is consumed by the hydroxylase catalyzing the hydroxylation reaction, and the enzyme activity can be measured from the consumption of oxygen by, for example, an electrochemical method using an oxygen electrode.

The reduced mediator (also referred to as a proteinaceous mediator, an artificial electron mediator, an artificial electron acceptor, or an electron mediator) used in the quantitation method, the hydroxylase, the quantification composition, the quantification kit, the electrode, the sensor chip, and the sensor of the present invention is not particularly limited as long as it can supply electrons to the hydroxylase. Examples of the mediator include quinones, phenazines such as safranins, viologens, cytochromes, phenoxazines such as nile blue, phenothiazines, ferricyanides such as potassium ferrocyanide, ferredoxins, isoalloxazines such as riboflavin, ferrocene, syringaldazine, 2,2′-azino-bis(3-ethylbenzothiazoline-6-sulfonic acid), osmium complexes, ruthenium complexes, and derivatives thereof.

Compounds of the phenothiazines include, but are not limited to, Azur A, Azur B, Azur C, thionine acetate, methylene green, methylene blue, 10-(carboxymethylaminocarbonyl)-3,7-bis(dimethylamino)-phenothiazine (DA-67). The mediator of a low molecular weight compound may be a light-absorbing substance, in which case the enzyme activity can be measured by performing the absorbance measurement.

Proteinaceous mediators of ferredoxins include, but are not limited to, ferredoxin reduced by ferredoxin reductase. The ferredoxin reductase receives electrons from NADH or NADPH and reduces ferredoxin. The reduced ferredoxin supplies electrons to the hydroxylase, and the hydroxylase supplied with electrons to the heme iron which hydroxylates 25-hydroxyvitamin D. Enzyme activity can be measured by reacting a light-absorbing reagent containing a light-absorbing material (in this case, NADH or NADPH) reacting with a proteinaceous mediator and performing the absorbance measurement.

In an embodiment, the ferredoxin includes ferredoxin derived from Spinacia oleracea, but the present invention is not limited to this, and includes ferredoxin that can supply electrons to the hydroxylase that hydroxylates 25-hydroxyvitamin D. In an embodiment, the ferredoxin of the present invention may be a ferredoxin derived from a microorganism classified into Hordeum vulgare, Lactuca saligna, Corymbia citriodora, Rhododendron griersonianum, Beta vulgaris, Mesembryanthemum crystallinum, Medicago sativa, Colocasia esculenta, Alocasia macrorrhizos, Triticum turgidum, Mycobacterium tuberculosis, Eucalyptus grandis, Chenopodium quinoa, Lactuca sativa, Rosa chinensis, Papaver somniferum, Prosopis alba, Syzygium oleosum, Leucaena leucocephala, Corchorus capsularis, and Impatiens glandulifera. In addition, the ferredoxin can include a ferredoxin which has high sequence identity (e.g., 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, and 99% or more) relative to the amino acid sequence of ferredoxin in SEQ ID NO: 3, and a ferredoxin having an amino acid sequence in which one or more amino acids are altered or varied, deleted, substituted, added and/or inserted in the amino acid sequence of SEQ ID NO: 3.

In an embodiment, the ferredoxin reductase includes a ferredoxin reductase derived from Spinacia oleracea, but the present invention is not limited to this, and includes a ferredoxin reductase that reduces the ferredoxin. In an embodiment, the ferredoxin reductase of the present invention may be a ferredoxin reductase derived from a microorganism classified into the genus Abrus, the genus Acer, the genus Aquilegia, the genus Arachis, the genus Asparagus, the genus Benincasa, the genus Beta, the genus Cajanus, the genus Camellia, the genus Capsicum, the genus Carpinus, the genus Castanea, the genus Cephalotus, the genus Chenopodium, the genus Cicer, the genus Citrus, the genus Coffea, the genus Colocasia, the genus Corchorus, the genus Cucumis, the genus Cucurbita, the genus Durio, the genus Erythranthe, the genus Eucalyptus, the genus Glycine, the genus Gossypium, the genus Herrania, the genus Hevea, the genus Hibiscus, the genus Ipomoea, the genus Jatropha, the genus Juglans, the genus Kingdonia, the genus Macleaya, the genus Malus, the genus Manihot, the genus Medicago, the genus Mesembryanthemum, the genus Momordica, the genus Morus, the genus Mucuna, the genus Nelumbo, the genus Nicotiana, the genus Nyssa, the genus Olea, the genus Papaver, the genus Parasponia, the genus Phaseolus, the genus Phtheirospermum, the genus Pistacia, the genus Pisum, the genus Populus, the genus Prosopis, the genus Prunus, the genus Punica, the genus Quercus, the genus Rhamnella, the genus Rhodamnia, the genus Ricinus, the genus Rosa, the genus Salix, the genus Salvia, the genus Solanam, the genus Spatholobus, the genus Striga, the genus Syzygium, the genus Theobroma, the genus Trema, the genus Trifolium, the genus Vicia, the genus Vigna, the genus Vitis, the genus Ziziphus, the genus Heliosperma, the genus Lithospermum, the genus Buddleja, the genus Kalanchoe, the genus Actinidia, the genus Thalictrum, the genus Tripterygium, the genus Lupinus, the genus Arabidopsis, the genus Arabis, the genus Capsella, the genus Daucus, the genus Musa, the genus Apostasia, the genus Eutrema, the genus Dendrobium, the genus Raphanus, the genus Ananas, the genus Brassica, the genus Microthlaspi, the genus Amborella, the genus Dorcoceras, the genus Mikania, the genus Artemisia, the genus Cuscuta, the genus Helianthus, the genus Eragrostis, the genus Zostera, the genus Oryza, the genus Lactuca, the genus Triticum, the genus Aegilops, the genus Brachypodium, the genus Dichanthelium, the genus Zea, the genus Setaria, the class Nitriliruptoraceae, and the genus Sorghum. In addition, in an embodiment, the ferredoxin reductase of the present invention may be a ferredoxin reductase derived from a microorganism classified into Actinidia chinensis var. chinensis, Cinnamomum micranthum f. kanehirae, Escherichia coli, Mentha x piperita, Pyrus ussuriensis x Pyrus communis, Vigna radiata var. radiata, Pyrus x bretschneideri, Juglans microcarpa x Juglans regia, Camellia sinensis var. sinensis, Prunus yedoensis var. nudiflora, Elaeis guineensis var. tenera, Brassica oleracea var. oleracea, and Panicum hallii var. hallii. In addition, the ferredoxin reductase can include a ferredoxin reductase which has high sequence identity (e.g., 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, and 99% or more) relative to the amino acid sequence of ferredoxin reductase in SEQ ID NO: 4, and a ferredoxin reductase having an amino acid sequence in which one or more amino acids are altered or varied, deleted, substituted, added and/or inserted in the amino acid sequence of SEQ ID NO: 4.

### [Method for Quantifying 25-Hydroxyvitamin D]

The method for quantifying 25-hydroxyvitamin D may be any method as long as it is a method for directly or indirectly measuring the product or consumption of the hydroxylation reaction of 25-hydroxyvitamin D catalyzed by hydroxylase. For example, a calibration curve can be generated by measuring enzyme activity for known 25-hydroxyvitamin D concentrations and plotting against 25-hydroxyvitamin D concentrations. The concentration of 25-hydroxyvitamin D can be obtained from the calibration curve by measuring the activity of an unknown 25-hydroxyvitamin D enzyme.

In an embodiment, in the case of quantifying 25-hydroxyvitamin D using blood as a sample, the sample can be optionally selected from whole blood, plasma, or serum, depending on the 25-hydroxyvitamin D to be measured. In addition, the quantification composition of 25-hydroxyvitamin D containing hydroxylase may be mixed directly with the sample, and the sample may be pretreated before mixing with the quantification composition of 25-hydroxyvitamin D containing hydroxylase. For example, the vitamin D binding protein may be degraded with a protease to liberate 25-hydroxyvitamin D and then mixed with the quantification composition of 25-hydroxyvitamin D containing hydroxylase.

[Composition Containing Hydroxylase and Quantification Kit for 25-Hydroxyvitamin D]

The method for quantifying 25-hydroxyvitamin D using hydroxylase according to the present invention may be performed by providing a composition containing hydroxylase and a reaction reagent, or may be performed by combining hydroxylase and a commercially available reaction reagent. For example, the present invention may be provided as a composition for quantifying 25-hydroxyvitamin D containing hydroxylase, a composition for quantifying 25-hydroxyvitamin D further containing a mediator oxidized by the addition of hydroxylase, or a composition for quantifying 25-hydroxyvitamin D further containing a mediator oxidized by the addition of hydroxylase and a reagent reacting with the oxidized mediator. In addition, the present invention may be provided as a kit for quantifying 25-hydroxyvitamin D containing hydroxylase and a mediator oxidized by the addition of a hydroxylase, or a kit for quantifying 25-hydroxyvitamin D containing hydroxylase, a mediator oxidized by the addition of a hydroxylase, and a reagent reacting with the oxidized mediator.

The method for quantifying 25-hydroxyvitamin D, the hydroxylase for quantification, the quantification composition, and the quantification kit according to the present invention can provide a novel method for quantifying 25-hydroxyvitamin D, hydroxylase for quantification, a quantification composition, and a quantification kit which is an indicator of diseases associated with vitamin D deficiency by containing hydroxylase and a reduced mediator.

### [Sensor Chip and Electrode]

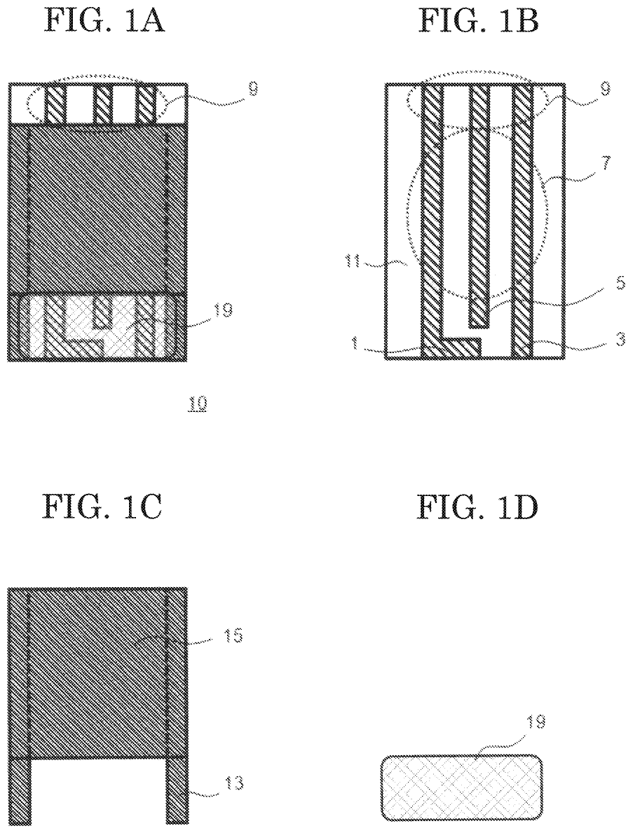

FIG. 1A is a schematic diagram of a sensor chip 10 according to an embodiment of the present invention, and FIG. 1B to FIG. 1D are schematic diagrams showing members constituting the sensor chip 10. The sensor chip 10 includes two or more electrodes arranged on a base material 11. The base material 11 is composed of an insulating material. In FIG. 1A and FIG. 1B, as an example, a working electrode 1, a counter electrode 3, and a reference electrode 5 are arranged on the base material 11. Each electrode is electrically connected to a wiring part 7, and the wiring part 7 is electrically connected to a terminal 9 arranged opposite to each electrode in the wiring direction. The working electrode 1, the counter electrode 3, and the reference electrode 5 are spaced apart from each other. In addition, the working electrode 1, the counter electrode 3, and the reference electrode 5 are preferably formed integrally with the wiring part 7 and the terminal 9. Further, the counter electrode 3 and the reference electrode 5 may be integrated.

As shown in FIG. 1A and FIG. 1C, a spacer 13 is arranged at an end portion of the base material 11 parallel to the wiring part 7, and a cover 15 covering the working electrode 1, the counter electrode 3, the reference electrode 5, and the spacer 13 is arranged. The spacer 13 and the cover 15 are composed of an insulating material. The spacer 13 preferably has a thickness substantially equal to that of the working electrode 1, the counter electrode 3, and the reference electrode 5, and is in close contact with the working electrode 1, the counter electrode 3, and the reference electrode 5. In addition, the spacer 13 and the cover 15 may be integrally formed. The cover 15 is a protective layer that prevents the wiring part 7 from being deteriorated by being exposed to the outside air and short-circuited due to the penetration of the measurement sample.

In an embodiment, the hydroxylase of the present invention may be applied, adsorbed, or immobilized on the electrode. Preferably, the hydroxylase of the present invention is applied, adsorbed, or immobilized on the working electrode. In another embodiment, the mediator may also be applied, adsorbed, or immobilized on the electrode along with the hydroxylase. The hydroxylase or the hydroxylase and the mediator may be contained in a reaction layer arranged on the working electrode, the counter electrode, and the reference electrode. A carbon electrode, a metal electrode such as platinum, gold, silver, nickel, and palladium, or the like can be used as the electrode. In the case of the carbon electrode, materials include pyrolytic graphite carbon (PG), glassy carbon (GC), carbon pastes, plastic foamed carbon (PFC), and the like. A measurement system may be a two-electrode system or a three-electrode system, for example, an enzyme can be immobilized on the working electrode. Examples of the reference electrode include a standard hydrogen electrode, a reversible hydrogen electrode, a silver-silver chloride electrode (Ag/AgCl), a palladium-hydrogen electrode, and a saturated calomel electrode, and Ag/AgCl is preferably used from the viewpoint of stability and reproducibility.

The enzyme may be immobilized to the electrode by crosslinking, coating with a dialysis membrane, encapsulation in a polymeric matrix, use of a photocrosslinkable polymer, use of an electrically conductive polymer, or use of an oxidizing/reducing polymer, and the like. In addition, an enzyme may be immobilized in a polymer or adsorbed onto an electrode with a mediator, and these techniques may be combined.

The hydroxylase of the present invention can be applied to various electrochemical measurement methods by using potentiostats, galvanostats, or the like. The electrochemical measurement methods include various techniques such as amperometry, potentiometry, and coulometry. For example, a mediator is mixed into a reaction solution and when −400 mV to +500 mV (vs. Ag/AgCl) is applied by an amperometric method, the reduced mediator supplies electrons to the hydroxylase to generate an oxidized mediator, and the concentration of 25-hydroxyvitamin D in a sample can be calculated by measuring a current value generated when hydroxylase that has received electrons reacts with 25-hydroxyvitamin D. The counter electrode is preferably a carbon electrode or a platinum electrode. For example, a calibration curve can be created by measuring current values for known 25-hydroxyvitamin D concentrations (0 mM to 0.5 mM) and plotting against 25-hydroxyvitamin D concentrations. The concentration of 25-hydroxyvitamin D can be obtained from the calibration curve by measuring a current value of unknown 25-hydroxyvitamin D.

In addition, a printed electrode (sensor chip) can also be used to reduce the amount of solution required for the measurement. In this case, the electrode is preferably formed on a base material composed of an insulating substrate. Specifically, it is desirable that the electrode be formed on the base material by a photolithography technique and a printing technique such as screen printing, gravure printing, and flexographic printing. Examples of the material of the insulating substrate include silicones, glasses, ceramics, polyvinyl chloride, polyethylenes, polypropylenes, and polyesters, and it is more preferable to use materials having high resistance to various solvents and chemicals.

### [25-Hydroxyvitamin D Measurement Sensor]

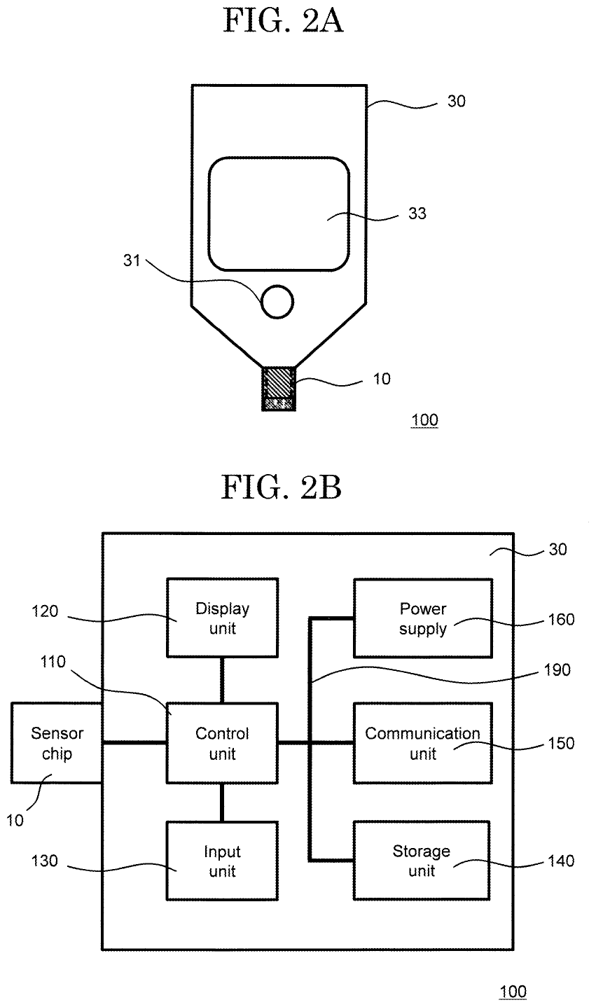

In an embodiment, a 25-hydroxyvitamin D measurement sensor using the hydroxylase of the invention is provided. FIG. 2A is a schematic diagram of a sensor 100 according to an embodiment of the present invention. The sensor is a 25-hydroxyvitamin D measurement device using the hydroxylase of the present invention, and includes a sensor chip containing hydroxylase and a measurement unit. A measurement unit 30 may include, for example, a switch 31 as an input unit and a display 33 as a display unit. For example, the switch 31 may be used to control ON/OFF of a power source of the measurement unit 30, or may be used to control the initiation or interruption of the 25-hydroxyvitamin D measurement by the sensor 100. For example, the display 33 may display a measurement value of 25-hydroxyvitamin D, and may include a touch panel as an input unit for controlling the measurement unit 30.

FIG. 2B is a block diagram of the sensor 100 according to an embodiment of the present invention. For example, the sensor 100 may include a control unit 110, a display unit 120, an input unit 130, a storage unit 140, a communication unit 150, and a power supply 160 in the measurement unit 30, which may be electrically connected to each other by a wiring 190. In addition, a terminal of the sensor chip 10 and a terminal of the measurement unit 30, which will be described later, are electrically connected to each other, and a current generated in the sensor chip 10 is detected by the control unit 110. The control unit 110 is a control device for controlling the sensor 100, and configured by, for example, a known central processing unit (CPU) and an operation program for controlling the sensor 100. Alternatively, the control unit 110 includes a central processing unit and an operating system (OS) and may include an application program or module for performing the 25-hydroxyvitamin D measurement.

For example, the display unit 120 includes a known display 33, and may display a measurement value of 25-hydroxyvitamin D, a condition of the measurement unit 30, and an operation request to a measurer. The input unit 130 is an input device for the measurer to operate the sensor 100, and may be, for example, the switch 31 or a touch panel arranged in the display 33. A plurality of switches 31 may be arranged in the measurement unit 30.

The storage unit 140 is composed of a main storage device (memory), and an auxiliary storage device (hard disk) may be arranged externally. The main storage device (memory) may be composed of a read-only memory (ROM) and/or a random access memory (RAM). The operation program, the operating system, the application program, or the module are stored in the storage unit 140 and executed by the central processing unit to constitute the control unit 110. In addition, the measured value and the current value can be stored in the storage unit 140.

The communication unit 150 is a known communication device that connects the sensor 100 or the measurement unit 30 to an external device (a computer, a printer, or a network). The communication unit 150 and the external device are connected by wired or wireless communication. In addition, the power supply 160 is a known power supply device that supplies power to the sensor 100 or the measurement unit 30.

As described above, the method for quantifying 25-hydrocyvitamin D, hydroxylase for quantification, the quantification composition, the quantification kit, the electrode, the sensor chip, and the sensor according to the present invention may provide a novel quantification method for quantifying the 25-hydrocyvitamin D concentration, hydroxylase, the quantification composition, the quantitative kit, the electrode, the sensor chip, and the sensor by containing hydroxylase and the reduced mediator.

### Example 1

Specific examples and test results of the method for quantifying 25-hydroxyvitamin D, the hydroxylase for quantification, the quantification composition, and the quantification kit according to the present invention described above will be described in more detail.

[Preparation of Recombinant Plasmid pET-16b_VDH]

A VDH gene derived from a Pseudonocardia autotrophica NBRC12743 strain having the base sequence of SEQ ID NO: 2 was codon-optimized for E. coli., divided into the first half (vdh-f1) of SEQ ID NO: 5 and the second half (vdh-f2) of SEQ ID NO: 6, and the synthesis was entrusted to Integrated DNA Technologies. 15 bases at the 3′ end of vdh-f1 and 15 bases at the 5′ end of vdh-f2 (TGACTTCTCCGCGTG) represent the sequence of SEQ ID NO: 9 that overlaps the first half and the second half of the vdh gene.

A DNA fragment of the vector pET-16b was amplified by PCR using primers of SEQ ID NOs: 7 and 8 using pET-16b as a template. 1.0 μl of DpnI (manufactured by New England BioLabs) was added to a PCR solution and treated at 37° C. for 1 hour, and then subjected to agarose gel electrophoresis to cut out a gel containing a desired DNA fragment (about 5.7 kbp). The desired DNA fragment was extracted from the gel using a QIAquick Gel Extraction Kit (manufactured by QIAGEN).

A plasmid for expression (pET-16b_VDH) of VDH with a histidine tag removed was obtained using the DNA fragment of vector pET-16b and two VDH gene fragments (vdh-f1 and vdh-f2), and performing an in-fusion reaction (50° C., 15 min) according to the manuals of In-Fusion (registered trademark) HD Cloning Kit (manufactured by Takara Bio Inc.). An E. coli BL21 (DE3) strain was transformed with the obtained plasmid.

### [VDH Expression]

The E. coli BL21 (DE3) strain having the recombinant plasmid (pET-16b_VDH) was inoculated into 2.5 ml of an LB-amp medium [1% (W/V) Bactotryptone, 0.5% (W/V) yeast extract, 0.5% (W/V) NaCl, 50 μg/ml Ampicillin], and cultured with shaking at 37° C. overnight to obtain a culture.

4 tubes of 2.5 ml of the seed culture solution were added to 500 ml of a MCG-amp medium [0.54% (w/v) disodium hydrogen phosphate, 0.3% (w/v) potassium dihydrogen phosphate, 0.05% (w/v) sodium chloride, 0.1% (w/v) ammonium chloride, 1% (w/v) casamino acid, 0.4% (w/v) glucose, 980 μM magnesium chloride, 1.35 mM calcium chloride, 100 μM ferric sulfate, 50 μg/ml Ampicillin], and then cultured with shaking at 37° C. for 2 and a half hours. Subsequently, 15 ml of 99.5% ethanol and 50 ml of 50% (w/w) glycerol per 500 ml of MCG medium were added to the medium and cultured with shaking at 22° C. for 20 minutes. Further, IPTG was added so as to be a final concentration of 100 μM and 5-aminolevulinic acid was added so as to be 80 μg/ml and cultured with shaking at 22° C. for 20 hours.

Bacteria were collected by centrifugation of the culture solution at 5600×g for 10 minutes. The obtained bacterial cells were resuspended in 50 mM of a potassium phosphate buffer (pH 7.4) containing 10% (v/v) glycerol and 2 mM dithiothreitol. The bacterial cell suspension was disrupted by ultrasonication, and then centrifuged at 5600×g for 10 minutes, and the supernatant was used as a crude enzyme solution.

The crude enzyme solution was transferred to an Amicon (registered trademark) Ultra Ultracel-30K (manufactured by Merck), centrifuged at 5600×g for 15 minutes, and concentrated.

### [Purification of VDH]

Ammonium sulfate was added to a VDH crude enzyme solution so as to have a 35% saturated concentration, thoroughly vortexed to dissolve ammonium sulfate, and centrifuged at 5600×g for 10 minutes to obtain a supernatant. The obtained supernatant fraction was filter-sterilized with 0.20 μm Minisart (registered trademark) Syringe Filter (manufactured by Sartorius). The fraction was fractionated with HiLoad 26/60 Superdex 200 (manufactured by GE Healthcare) equilibrated with 10 mM of a potassium phosphate buffer (pH 7.4) containing 150 mM NaCl. The purity of the eluted fractions was assessed by polyacrylamide gel electrophoresis (SDS-PAGE), and the fractions free of contaminated proteins were collected to obtain a purified preparation of VDH. The purified preparation was used by replacing with 50 mM of a potassium phosphate buffer containing 10% (v/v) glycerol (pH 7.4) using the Amicon (registered trademark) Ultra Ultracel-30K.

The protein concentration of the purified VDH was measured by a UV absorption method using absorbance (A280) at 280 nm (see Protein Sci. 4, 2411-23, 1995). Since the molecular weight of VDH calculated from the amino acid sequence is 45.0 kDa, and since VDH contains 6 tyrosine residues and 5 tryptophan residues, A280 of 1.0 mg/ml VDH solution indicates 0.810.

### [Quantification of 25-Hydroxyvitamin D by Hydroxylase Activity Using DA-67 as a Mediator]

VDH obtained by the above-described methods and DA-67 were used to measure hydroxylase activity using 25-hydroxyvitamin D3, a vitamin D derivative, as a substrate.

### [Substrate Reagent]

4.19 mg of 25-hydroxyvitamin D3 was dissolved in 1 ml of DMSO (final concentration: 10 mM). Since 25-hydroxyvitamin D3 is lipid-soluble, it was diluted to 20 μM, 50 μM, and 100 μM (concentration at the time of measurement: 10 μM, 25 μM, and 50 μM) using an approximately 3.9% (v/v) TritonX-100 solution to maintain solubility.

TABLE 1
61.8 μM hydroxylase
112 mM sodium chloride
80 mM Tris-hydroxymethylaminomethane-hydrochloric acid buffer
(pH 7.5)
0.2% γ-cyclodextrin (manufactured by Fujifilm Wako Pure Chemical
Corporation)
0.05% partially methylated B-cyclodextrin (manufactured by Sigma-
Aldrich)
0.1 mM DA-67 (manufactured by Fujifilm Wako Pure Chemical
Corporation)

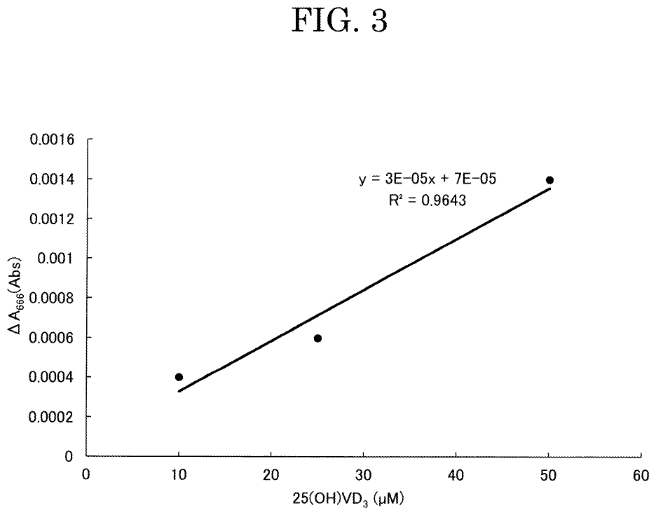

After incubating 375 μl of the measurement reagent consisting of the components of Table 1 at 30° C. for 3 minutes, 375 μl of the substrate reagent was added, and the absorbance (Abs) at the wavelength 666 nm was measured over time for 360 seconds using a spectrophotometer (U-3900, manufactured by Hitachi High-Tech Science Corporation). The value calculated by dividing the amount of change in absorbance for 300 seconds from 60 seconds to 360 seconds after the start of measurement by 5 was obtained as the amount of change in absorbance per minute. The relationship between the 25-hydroxyvitamin D concentration and the amount of change in absorbance per minute (ΔA666) is shown in FIG. 3.

Since VDH showed the coefficient of determination (R²) of 0.964 in a range of 10 μM to 50 μM, which is an index of a correlation between 25-hydroxyvitamin D concentration and absorbance, the result showed that there is a correlation between 25-hydroxyvitamin D concentration and absorbance. Therefore, it was shown that 25-hydroxyvitamin D can be quantified by hydroxylase activity of VDH using DA-67 as a mediator.

### Example 2

### [Quantification of 25-Hydroxyvitamin D by Hydroxylase Activity Using Ferredoxin as a Mediator]

VDH obtained by the above-described method and ferredoxin were used to measure hydroxylase activity using 25-hydroxyvitamin D3, which is a vitamin D derivative, as a substrate.

### [Substrate Reagent]

4.19 mg of 25-hydroxyvitamin D3 was dissolved in 1 ml of DMSO (final concentration: 10 mM). Since 25-hydroxyvitamin D3 is lipid-soluble, it was diluted to 1 mM (concentration at the time of measurement: 0.5 mM) using 4.0% (v/v) TritonX-100 solution to maintain solubility.

TABLE 2
2.00 μM hydroxylase
100 mM sodium chloride
81.4 mM Tris-hydroxymethylaminomethane-hydrochloric acid buffer
(pH 7.5)
96 μg/ml ferredoxin derived from Spinacia oleracea (manufactured by
Sigma-Aldrich)
0.1 U/ml ferredoxin reductase derived from Spinacia oleracea
(manufactured by Sigma-Aldrich)
0.2 mM nicotinamide adenine nucleotide phosphate (NADPH)
(manufactured by Oriental Yeast Co., Ltd.)
Milli-Q

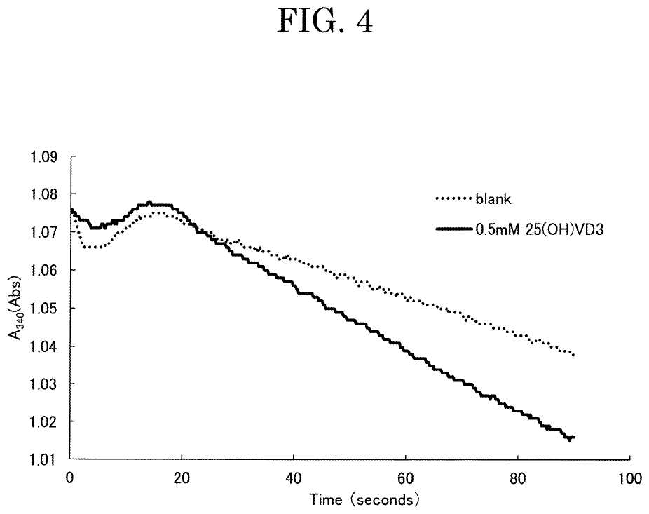

After incubating 500 μl of the measurement reagent consisting of the composition of Table 2 at 30° C. for 3 minutes, 500 μl of the substrate reagent was added, and the absorbance (Abs) at the wavelength 340 nm was measured over time for 90 seconds using a spectrophotometer (U-3900, manufactured by Hitachi High-Tech Science Corporation). FIG. 4 shows a reaction time and absorbance (A340) of samples of 0 mM 25(OH)VD₃ (blank) and 0.5 mM 25(OH)VD₃. VDH showed a significant change in absorbance in the presence of 0.5 mM 25-hydroxyvitamin D compared to the blank. Therefore, it was shown that 25-hydroxyvitamin D can be quantified by hydroxylase activity of VDH using ferredoxin as a mediator.

As described above, a method for quantifying 25-hydroxyvitamin D by adding hydroxylase to a sample containing 25-hydroxyvitamin D and a mediator according to the present invention, a hydroxylase for quantifying 25-hydroxyvitamin D added to a sample containing 25-hydroxyvitamin D and a mediator, a composition for quantifying 25-hydroxyvitamin D containing hydroxylase and a mediator, and a kit for quantifying 25-hydroxyvitamin D containing hydroxylase added to a sample containing 25-hydroxyvitamin D can provide a novel method for quantifying a 25-hydroxyvitamin D concentration, a hydroxylase for quantification, a quantification composition, a quantification kit, an electrode, a sensor chip, and a sensor.

What is claimed is:

1. A method for quantifying 25-hydroxyvitamin D comprising:
adding a mediator and hydroxylase to a sample.

2. The method for quantifying 25-hydroxyvitamin D according to claim 1, comprising:
quantifying an oxidized mediator produced by an action of the hydroxylase, or oxygen or a reduced mediator consumed by an action of the hydroxylase.

3. A hydroxylase used in the method for quantifying 25-hydroxyvitamin D according to claim 1.

4. A composition for quantifying 25-hydroxyvitamin D comprising:
the hydroxylase according to claim 3.

5. A kit for quantifying 25-hydroxyvitamin D comprising:
the hydroxylase according to claim 3, and
a mediator oxidized by adding the hydroxylase.

6. An electrode comprising:
the hydroxylase according to claim 3.

7. A sensor chip comprising:
the electrode according to claim 6 as a working electrode.

8. A sensor comprising:
the sensor chip according to claim 7.


SEQUENCE LISTING (9 sequences)

SEQ ID NO: 1 - protein, 403 aa, Pseudonocardia autotrophica
MALTTTGTEQHDLFSGTFWQNPHPAYAALRAEDPVRKLALPDGPVWLLTRYADVREAFVDPRLSKDWRHTLPEDQRADMPATPTPMMILMDPPDHTRLRKLVGRSFTVRRMNELEPRITEIADGLLAGLPTDGPVDLMREYAFQIPVQVICELLGVPAEDRDDFSAWSSVLVDDSPADDKNAAMGKLHGYLSDLLERKRTEPDDALLSSLLAVSDEDGDRLSQEELVAMAMLLLIAGHETTVNLIGNGVLALLTHPDQRKLLAEDPSLISSAVEEFLRFDSPVSQAPIRFTAEDVTYSGVTIPAGEMVMLGLAAANRDADWMPEPDRLDITRDASGGVFFGHGIHFCLGAQLARLEGRVAIGRLFADRPELALAVGLDELVYRESTLVRGLSRMPVTMGPRSA

SEQ ID NO: 2 - DNA, 1212 bp, Pseudonocardia autotrophica
  source 1..1212: mol_type = genomic DNA
atggcattgacaactaccggaactgaacagcacgatttgttctcaggcacgttctggcagaatccccaccccgcgtatgctgccctgcgtgcggaggatcccgttcgcaagttggctttacccgatgggccggtctggcttttgacacgttacgccgatgtacgtgaggcattcgtagatccgcgcttgtctaaggactggcgtcataccctgccagaagaccagcgtgctgacatgcccgccaccccaacgcccatgatgattttaatggacccgcccgatcatacacgcctgcgcaagcttgtgggtcgttcatttaccgttcgccgtatgaatgaacttgaaccccgcattacagaaatcgctgatggattacttgctgggttgccaactgacggacccgtagatttgatgcgtgagtacgctttccaaatcccagtgcaagtgatttgcgaattactgggcgtgcccgctgaagatcgcgatgacttctccgcgtggtcatccgtgttagttgatgactcaccagctgacgacaagaatgctgctatgggcaagttacacggatacctgtccgacttgttggaacgcaagcgtacggaaccagacgacgctcttttgagtagtttactggccgtgagtgacgaagacggtgaccgccttagtcaagaagaattagtcgctatggcgatgctgttgttaattgcggggcacgagaccacagtaaacttaatcgggaatggcgtgctggctcttctgacgcaccccgaccaacgtaagctgcttgccgaagacccgtctttgatttcgagtgcagtcgaggagtttttgcgctttgatagtccagtaagccaggcacccattcgttttaccgctgaggatgtgacatactctggtgtcaccattcccgcaggagaaatggtcatgttaggtttggccgcagctaatcgcgatgcggattggatgccagagccggaccgtttggacatcactcgtgatgcttcgggcggagtgttttttggacatggtatccatttctgtctgggcgcccaattagcacgtttggagggacgtgtcgctattggtcgtttattcgccgatcgccctgagcttgcgttggctgtaggactggatgagttagtataccgtgaatctacgttagttcgcggattgtctcgcatgcccgtgacgatgggaccacgcagcgcataa

SEQ ID NO: 3 - protein, 98 aa, Spinacia oleracea
MAAYKVTLVTPTGNVEFQCPDDVYILDAAEEEGIDLPYSCRAGSCSSCAGKLKTGSLNQDDQSFLDDDQIDEGWVLTCAAYPVSDVTIETHKEEELTA

SEQ ID NO: 4 - protein, 315 aa, Spinacia oleracea
MQIASDVEAPPPAPAKVEKHSKKMEEGITVNKFKPKTPYVGRCLLNTKITGDDAPGETWHMVFSHEGEIPYREGQSVGVIPDGEDKNGKPHKLRLYSIASSALGDFGDAKSVSLCVKRLIYTNDAGETIKGVCSNFLCDLKPGAEVKLTGPVGKEMLMPKDPNATIIMLGTGTGIAPFRSFLWKMFFEKHDDYKFNGLAWLFLGVPTSSSLLYKEEFEKMKEKAPDNFRLDFAVSREQTNEKGEKMYIQTRMAQYAVELWEMLKKDNTYVYMCGLKGMEKGIDDIMVSLAAAEGIDWIEYKRQLKKAEQWNVEVY

SEQ ID NO: 5 - DNA, 500 bp, synthetic construct
  misc_feature 1..500: note = vdh-f1
  source 1..500: mol_type = other DNA
atggcattgacaactaccggaactgaacagcacgatttgttctcaggcacgttctggcagaatccccaccccgcgtatgctgccctgcgtgcggaggatcccgttcgcaagttggctttacccgatgggccggtctggcttttgacacgttacgccgatgtacgtgaggcattcgtagatccgcgcttgtctaaggactggcgtcataccctgccagaagaccagcgtgctgacatgcccgccaccccaacgcccatgatgattttaatggacccgcccgatcatacacgcctgcgcaagcttgtgggtcgttcatttaccgttcgccgtatgaatgaacttgaaccccgcattacagaaatcgctgatggattacttgctgggttgccaactgacggacccgtagatttgatgcgtgagtacgctttccaaatcccagtgcaagtgatttgcgaattactgggcgtgcccgctgaagatcgcgatgacttctccgcgtg

SEQ ID NO: 6 - DNA, 727 bp, synthetic construct
  misc_feature 1..727: note = vdh-f2
  source 1..727: mol_type = other DNA
tgacttctccgcgtggtcatccgtgttagttgatgactcaccagctgacgacaagaatgctgctatgggcaagttacacggatacctgtccgacttgttggaacgcaagcgtacggaaccagacgacgctcttttgagtagtttactggccgtgagtgacgaagacggtgaccgccttagtcaagaagaattagtcgctatggcgatgctgttgttaattgcggggcacgagaccacagtaaacttaatcgggaatggcgtgctggctcttctgacgcaccccgaccaacgtaagctgcttgccgaagacccgtctttgatttcgagtgcagtcgaggagtttttgcgctttgatagtccagtaagccaggcacccattcgttttaccgctgaggatgtgacatactctggtgtcaccattcccgcaggagaaatggtcatgttaggtttggccgcagctaatcgcgatgcggattggatgccagagccggaccgtttggacatcactcgtgatgcttcgggcggagtgttttttggacatggtatccatttctgtctgggcgcccaattagcacgtttggagggacgtgtcgctattggtcgtttattcgccgatcgccctgagcttgcgttggctgtaggactggatgagttagtataccgtgaatctacgttagttcgcggattgtctcgcatgcccgtgacgatgggaccacgcagcgcataa

SEQ ID NO: 7 - DNA, 30 bp, synthetic construct
  misc_feature 1..30: note = primer 1
  source 1..30: mol_type = other DNA
cacgcagcgcataaggatccggctgctaac

SEQ ID NO: 8 - DNA, 30 bp, synthetic construct
  misc_feature 1..30: note = primer 2
  source 1..30: mol_type = other DNA
agttgtcaatgccatggtatatctccttct

SEQ ID NO: 9 - DNA, 15 bp, synthetic construct
  misc_feature 1..15: note = 15 bases at the 3'end side of vdh-f1 and 15 bases at the 5'end side of vdh-f2
  source 1..15: mol_type = other DNA
tgacttctccgcgtg